(19) United States
(12) Patent Application Publication
(10) Pub. No.: US 2020/0030062 A1
(43) Pub. Date: Jan. 30, 2020

(54) IMPLANT SYSTEM

(71) Applicants: STRAUMANN HOLDING AG, Basel (CH); LAKEVIEW INNOVATION LTD., Buochs (CH)

(72) Inventors: Jochen KULLICK, Emmendingen (DE); Peter SAMSFORT, Endingen (DE); Andreas BORN, Reinach (CH); Christian PAATZ, Basel (CH)

(73) Assignees: STRAUMANN HOLDING AG, Basel (CH); LAKEVIEW INNOVATION LTD., Buochs (CH)

(21) Appl. No.: 16/496,316

(22) PCT Filed: Mar. 19, 2018
(86) PCT No.: PCT/EP2018/056851

§ 371 Date: Sep. 20, 2019

(30) Foreign Application Priority Data
Mar. 20, 2017 (EP) 17161897.8

(51) Int. Cl. A61C 8/00; A61C 13/00

(52) U.S. Cl. CPC A61C 8/0059; A61C 13/0006; A61C 8/0069; A61C 8/0054

(57) ABSTRACT

An implant system including a dental implant and an abutment produced from a ceramic material, wherein the abutment includes a first anti-rotation element with multiple grooves and the dental implant includes a second anti-rotation element, which is complementary thereto, with multiple ribs, wherein the grooves are open toward a proximal end of the abutment.

The present invention relates to an implant system including a dental implant and an abutment according to the preamble of claim 1.

Two-part or multi-part implant systems are well-established within the field of dental implantology and include, as a rule, a dental implant, which comprises an external thread, which is intended to be anchored in the bone of the patient, and an abutment (also called a secondary part), which serves as a base for the prosthetic structure. Frequently, in this case, the abutment is inserted into a corresponding coronal opening, i.e. facing the tooth crown in the implanted state, in the dental implant. The combination of separate dental implant and abutment is sometimes also designated in the literature as a “two-part (dental) implant”; in the present application the term “dental implant” simply designates the components to be anchored in the jaw bone (without the abutment).

The dental implant is produced in the majority of cases from metal, conventionally from titanium, titanium oxide, titanium alloys or the like. Above all, an implant system has to meet the highest quality requirements in terms of load-bearing capacity, functionality and service life. In this respect, great importance is given, in particular, to the mechanical connection between the two components of the implant system, i.e. to the connection between implant and abutment. Such a connection, in this case, has not only to ensure the receiving and forwarding of high chewing forces with the smallest dimensions, but must also enable play-free and anti-rotation positioning of the abutment in the dental implant. However, for the realization of an anti-rotation device between dental implant and abutment that is as positive-locking and play-free as possible, all mutual surface pairings have to be realized with accuracy of fit, which presupposes extremely precise production technology. Said problems have been met, for instance, in the following documents from the prior art:

U.S. Pat. No. 5,281,140 discloses a multi-part implant system with a two-part abutment. The latter includes a first part which is configured in such a manner at its lower end so as to be received in a complementary opening of the dental implant, and which comprises at its upper end a projection with a plurality of side faces in order to be received in a complementary opening of a second part of the abutment.

However, the solution described in said publication comprises, above all on account of the relatively large number of individual parts, disadvantages with regard to the stability of the connection between the abutment and the dental implant.

Proceeding from here, EP-A-1728486 has proposed an abutment for use in an implant system which comprises means for guiding and non-rotatably locking the abutment in the dental implant. Said means include a surface, which extends radially with reference to the axis of the abutment and is configured in such a way so as to interact with the dental implant in such a manner that the abutment is guided when inserted into the dental implant.

In addition, CA-A-2596988 describes an abutment which, in its apical region, comprises a groove, which forms an indexing element, for establishing the rotational position with respect to the dental implant.

Both the solution described in EP-A-1728486 and that described in CA-A-2596988 are geared for a traditional dental implant system produced on a metal base such as, for example, titanium.

As regards the abutment, for aesthetic reasons the longer it is, the more ceramic material is used. It has been shown that the gingiva and often also the jaw bone recede over the wearing period of the denture, as a result of which the metal dental implant becomes visible and, on account of its dark coloring, also visually perceptible. From an aesthetic viewpoint, all-ceramic systems are consequently particularly advantageous. However, the material of the implant system is sometimes exposed to relatively high levels of load, which, in particular in the case of ceramic materials, can result in problems on account of their lower bending strength and higher fracture susceptibility compared to metals. Thus, on the one hand, there is the risk of the parts of the implant system becoming damaged when the necessary anti-rotation elements are milled into the material of the implant or abutment in the course of production. On the other hand, material fractures can arise in the region of the anti-rotation elements when load peaks occur, for instance when the anti-rotation elements in the implant also serve as a contact surface for an insertion tool and/or when chewing forces act on the ceramic components at an angle with respect to the axis of the implant system. Said problems appear more frequently in conjunction with ceramic dental implant systems where the abutment and the dental implant are connected by means of a connecting screw which penetrates the abutment, as in the case of said systems, the wall thickness of the dental implant and/or of the abutment has to be reduced on account of the additional space required for the screw channel.

WO 2014/7091346 discloses, for example, a screw for fixing a ceramic abutment on a ceramic implant. The implant comprises an inner bore with a thread. The screw is, for example, produced from plastics material and is realized incongruently with respect to said thread. The incongruity results in cold welding being generated between the screw and the implant body such that a fixed seat is ensured for the screw. The disadvantage of said solution, however, is that the screw has to be bored out to release the connection between implant and abutment as, on account of the deformation of the screw body, reversible release is not possible.

Another system consisting of ceramic tooth implant and abutment with an implant screw is disclosed in EP-A-1529498. In the case of one embodiment, the implant screw comprises a conical contact surface and the abutment comprises a conical contact surface that is complementary thereto. The relative angular position between the implant screw and the abutment is, however, not fixed in the case of said embodiment.

In light of the abovementioned problems, the object to be achieved by the present invention consists in providing an implant system that includes a dental implant and an abutment produced from a ceramic material, the ceramic components of which can be connected in a non-rotatable manner in a certain relative position with regard to one another. At the same time, the connection is to enable good force transmission and to reduce the risk of material fractures.

The object is achieved according to the invention by an implant system according to claim 1. Preferred embodiments are the object of the dependent claims.

The implant system according to the present invention includes a dental implant and an abutment produced from a ceramic material. The dental implant is provided for anchoring in a jaw bone and extends in a longitudinal direction from an apical end to a coronal end. The shape of the dental implant is, as a rule, at least mirror-symmetrical with reference to its central longitudinal axis and it is normally realized in a (circular) cylindrical manner at least in portions, it being tapered in a preferred manner in the apical direction. According to the invention, the dental implant comprises an axial blind bore, which is open toward the coronal end, and additionally includes a threaded portion with a screw thread which is realized on an outer surface and in a preferred manner comprises a constant thread form. The dental implant can be screwed in a known manner into a bore hole in a jaw bone by means of the screw thread. To improve osteointegration characteristics, the dental implant can have been roughened on its surface additionally at least in regions and/or surface treated in another manner.

According to the invention, the abutment comprises a distal end with a head portion for receiving a prosthetic element, a connecting portion, which extends toward a proximal end, and a through-bore which extends in the longitudinal direction from the distal end up to the proximal end. The connecting portion is provided for insertion into the blind bore of the dental implant and, on the outside, comprises a first anti-rotation element. The first anti-rotation element is realized in a complementary manner to a second anti-rotation element which is realized on the inside in the blind bore of the dental implant.

According to the invention, the first anti-rotation element of the abutment includes a (hollow) cylindrical first basic body with an outer shell surface and additionally comprises multiple grooves which extend in the longitudinal direction and project from the outer shell surface into the first basic body, wherein said grooves are open toward the proximal end of the abutment. The second anti-rotation element of the dental implant includes a (hollow) cylindrical second basic body with an inner shell surface and multiple ribs which extend in the longitudinal direction and, proceeding from the inner shell surface, project into the axial blind bore. In the implanted state, the ribs of the dental implant engage in the grooves of the abutment and enable an anti-rotation connection between the two ceramic components of the implant system.

The choice of ribs projecting into the bind bore of the dental implant as an anti-rotation element according to the present invention has the advantage of the realization of the ribs not requiring any reduction in the wall thickness and consequently of no losses having to be accepted with reference to the stability of the dental implant. A high load-carrying capacity of the material in the region of the anti-rotation element is particularly important for the dental implant as the anti-rotation element thereof can also serve, in a preferred manner, as contact point or stop surface for a suitable insertion tool in order to anchor the implant in the jaw bone. In a known manner, in this case, a correspondingly formed free end of the insertion tool is moved into releasable engagement with the ribs of the second anti-rotation element in order to transmit a torsional moment to the dental implant. In contrast to known anti-rotation elements, which are milled or ground into the wall of the dental implant and consequently result in a reduced wall thickness at least in regions, according to the invention there is actually an increase in the wall thickness in the region of the second anti-rotation element on account of the ribs protruding into the interior of the blind bore (i.e. proceeding from the wall in the direction of the longitudinal axis of the dental implant) and this consequently makes transmission of greater torsional forces onto the dental implant possible.

Unlike the second anti-rotation element, the first anti-rotation element of the abutment serves primarily for realizing an anti-rotation connection between dental implant and abutment. The term “anti-rotation connection” is to be understood in this context as a state in which rotation of the abutment about the longitudinal axis in relation to the dental implant is prevented. When the first and second anti-rotation elements interact, the abutment can consequently be fixed in a certain alignment with reference to the dental implant. As the first anti-rotation element (in contrast to the second anti-rotation element) does not operate additionally according to the invention as a contact point for the transmission of torque, the reduced wall thickness in the region of the grooves is much less of a problem.

The choice of an anti-rotation device produced from interlocking grooves and ribs in the sense of the present invention has the advantage of little rotational play being ensured.

The two anti-rotation elements of the implant system include, in a preferred manner, an identical number of grooves or ribs. In a preferred manner, in each case at least three, in a more preferred manner in each case at least between four and eight, in a particularly preferred manner six ribs or grooves are provided. Said number ensures good force transmission from an insertion tool to the second anti-rotation element and good anti-rotation protection between abutment and dental implant. Too large a number of grooves, in contrast, weakens the material in the region of the first anti-rotation element, as there the grooves project into the basic body and consequently the wall thickness of the basic body in the region of the grooves is reduced by the depth of the grooves. The number of grooves/ribs certainly determines the number of alignment possibilities of the abutment in relation to the dental implant, however, from a certain number the marginal benefit for additional positioning possibilities is clearly reduced, whilst conversely the complexity with reference to the form of the anti-rotation elements increases. Consequently, a maximum of eight grooves/ribs are provided in a preferred manner.

As far as its geometric form is concerned, the grooves of the first anti-rotation element and the ribs of the second anti-rotation element each comprise, in a preferred manner, a cross section that is in the shape of a segmental arch at least in portions. This means that the cross-sectional surface of the grooves and ribs comprises at least one base line (as a rule lightly curved) which is formed through the outer or rather inner shell surface of the basic body of the respective anti-rotation element and the end points of which are connected by means of a connecting line which is arcuate at least in portions. In this case, it is possible to dispense with the realization of sharp edges and corners and, as a result, to avoid load peaks. In a preferred manner, said connecting line is in the shape of a circular arc at least in portions—in a particularly preferred manner completely—and, in the region of the circular arc, comprises a uniform radius, which allows forces acting on the respective anti-rotation element to be distributed in a homogeneous manner.

Irrespective of this, in a preferred manner, the anti-rotation elements of the implant or of the abutment are configured in such a manner that two adjacent grooves or ribs are spaced apart from one another in each case by portions of the inner or outer shell surface of the corresponding (hollow) cylindrical basic body. It is preferred that the (hollow) cylindrical basic body has a circular base area. In this connection too, it is possible to dispense with the realization of sharp edges and corners and, as a result, to avoid load peaks. Said portions between in each case two grooves or ribs ensure that the stability bestowed by the (hollow) cylindrical basic body is maintained in the region of the associated anti-rotation element. The width of the portions measured in the circumferential direction is preferably greater than the width of the grooves or ribs. This also means that the grooves and ribs are configured in a preferred manner so as to be rather narrow and elongated in each case with a steep gradient. Compared to a wide and flat configuration, in the case of the preferred narrower and deeper form, on the one hand the play between grooves and ribs in the connected state is reduced and, on the other hand, effective transmission of torsional moment from a corresponding insertion tool onto the ribs and consequently the dental implant is made possible. With regard to as good a transmission of torsional moment as possible, it is additionally preferred for the ribs (and in this respect also the grooves) to comprise a width-length ratio of between 1:3 and 1:6, in a preferred manner of approximately 1:4.

As mentioned further above, the abutment according to the invention includes a through-bore which is provided for receiving a connecting screw. In the case of a rotationally symmetrical abutment, the through-bore is arranged in a preferred manner along the longitudinal axis of the abutment. The abutment can, however, also comprise an angled form, which means that in the inserted state the longitudinal axis of the abutment and the longitudinal axis of the dental implant enclose an angle, whilst the axis of the through-bore is aligned as a rule with the longitudinal axis of the dental implant. In addition, it is conceivable for the through-bore not to be configured linearly but in a curved manner. This can be sensible in particular for abutments which are inserted into a dental implant placed right at the back of the mouth. As an alternative to this, this can also be suitable for abutments which can be placed in the anterior region in order, consequently, to ensure the outlet of the through-bore lingually.

By means of the connecting screw guided through the through-bore, the two ceramic parts can be connected together in a sturdy and positive locking manner such that, on the one hand, good force transmission from the abutment to the dental implant is achieved. The connecting screw is produced in a preferred manner from metal, in a preferred manner stainless steel, titanium or a titanium alloy as said materials ensure good stability, biocompatibility and sterilization. The additional advantage of metal materials is that they comprise a certain elasticity and the holding force of the connecting screw is increased as a result of the screw expanding minimally elastically along its longitudinal axis when screwed-in. The tensile force resulting from the expansion then results in a particularly sturdy connection between dental implant and abutment.

In the connected state of abutment and dental implant, the connecting screw engages in an internal threaded portion realized in the blind bore of the dental implant. The internal threaded portion can extend up to the apical end of the axial blind bore; in a preferred manner, however, it only extends over a part piece of the blind bore, as a result of which the production expenditure and also the time that is required for screwing-in the connecting screw is reduced. In a preferred manner, the internal threaded portion lies exclusively in the lower, i.e. apical, half of the blind bore. As a result, the length of the screw is increased, which increases the possible preload force of the screw.

According to a preferred embodiment, the blind bore comprises a (hollow) cylindrical end portion at the coronal end and consequently coronally of the second anti-rotation element, and the abutment comprises a complementary (hollow) cylindrical neck portion distally of the first anti-rotation element, the neck portion, once the two implant system components have been connected, being arranged inside the end portion and a precisely fitting connection being made possible. The second anti-rotation element consequently does not extend, in the case of said embodiment, up to the coronal end of the dental implant, but rather no further than up to the (hollow) cylindrical end portion. When, during chewing, with reference to its longitudinal axis, inclined forces act on the implant system, loads occur increasingly in particular in the region of the end portion of the implant and of the associated neck portion of the abutment. As the anti-rotation elements are arranged outside the end portion or the neck portion, the wall thickness in said regions is not additionally weakened and the end portion and neck portion are better able to absorb the forces that occur.

The (hollow) cylindrical end portion of the dental implant extends in a preferred manner substantially up to the coronal end of the axial blind bore. “Substantially” means in this connection that the end portion extends either completely up to the coronal end, or at least up to a point where the blind bore opens toward the coronal end (in particular, a shoulder-like transition region as a rule forms a gentle entry into the blind bore in order to avoid a sharp end edge). In a particularly preferred manner, the end portion extends in the coronal direction substantially up to the coronal end and in the apical direction up to the second anti-rotation element.

According to a preferred embodiment, the axial length of the end portion is at least half as long as the length of the second anti-rotation element. Analogously to this, the axial length of the neck portion is, in a preferred manner, at least half as long as the length of the first anti-rotation element. As a result, a more precisely fitting seat of the abutment in the dental implant is ensured, which once again reduces load peaks in the region of the first or second anti-rotation elements. In addition, it is ensured that the end portion or the neck portion is of sufficient length so as to prevent the abutment from tipping up when, during chewing, forces act at an angle on the implant system, with reference to the longitudinal axis thereof.

As mentioned above, in preferred embodiments the cylindrical end portion of the dental implant bore extends substantially to the coronal end of the bore, and similarly the complementary cylindrical neck portion of the abutment extends over the same length. In an alternative preferred embodiment however, the implant bore further comprises, coronal of the cylindrical end portion, a conically tapered section, the diameter of which increases in the coronal direction. In such embodiments the abutment connecting portion comprises, coronal of the cylindrical neck portion, a complementary conical tapered section such that, when the connecting portion of the abutment is fully inserted into the implant bore, the tapered surfaces are in contact with one another. The provision of such tapered contact surfaces provides an improved force transmission between the abutment and implant, as well as assisting with the centering of the abutment during connection to the implant.

Preferably the tapered portion of the implant bore and abutment post have a taper angle of between 5°-35°, more preferably 15-25° and most preferably approximately 20°.

Preferably the axial length of the tapered section of the implant bore, L_{I3}, is less than the axial length of the cylindrical end portion, L_{I2}, and the axial length of the second anti-rotation element, L_{I1}. Preferably LI₃ is less than L_{I2}, which in turn is less than L_{I1}. Particularly preferably, the axial length of L_{I3} is less than a fourth of LI₂, most preferably about a fifth of L_{I2}.

Similarly, preferably the axial length of the tapered section of the abutment, L_{A3}, is less than the axial length of the cylindrical neck portion, L_{A2}, and the axial length of the first anti-rotation element, L_{A1}. Preferably L_{A3} is less than L_{A2}, which in turn is less than L_{A1}. Particularly preferably, the axial length of L_{A3} is less than a fourth of L_{A2}, most preferably about a fifth of L_{A2}.

In embodiments featuring the above described tapered sections, the axial length of the end portion is reduced in comparison to embodiments which do not feature a tapered section, as the tapered section replaces a section of the cylindrical end portion. Therefore, in such embodiments the axial lengths of the end and neck portions may be less than half the length of the second and first anti-rotation elements, respectively. However, preferably the combined axial lengths of the tapered section and end portion are at least half as long as the axial length of the second anti-rotation element. Similarly, preferably the combined axial lengths of the tapered section and neck portion are at least half as long as the axial length of the first anti-rotation element.

In embodiments featuring the above tapered sections, it is further preferable that the connecting screw comprises a screw head having a conically tapered underside for abutment against a corresponding tapered screw seat in the through bore of the abutment. Such a tapered connection assists with the transmission of forces and furthermore directs the forces transmitted by the screw to the abutment towards the tapered sections of the bore and connecting portion.

Preferably the screw head has a taper angle of between 10° and 70°. In one preferred embodiment the screw head has a taper angle of between 10°-30°, most preferably 20°. In an alternative embodiment the screw head has a taper angle of between 50°-70°, most preferably 60°.

In a specifically preferred embodiment of the implant system, two complementary tapered sections are provided, one in the area of the blind bore of the implant and one in the area of the connecting portion of the abutment (i.e. a tapered section on the implant and a tapered section on the abutment), said tapered sections having a taper angle of approximately 20°; on the other hand, the screw head and the screw seat also comprise two complementary tapered sections (one tapered section on the screw head and one tapered section on the screw seat), which have a taper angle of either approximately 20° or approximately 60°.

As already mentioned, the dental implant comprises a threaded portion on the outside, the external thread of which extends over at least part of the dental implant. The external thread serves, in this case, for primary or immediate anchoring of the dental implant in a jaw bone. In a preferred manner, the threaded portion extends up to the apical end of the dental implant. As an alternative to this, the threaded portion extends at least over 50% of the overall length of the dental implant and preferably at least in the middle region of the dental implant. The external thread comprises, in a preferred manner, a uniform thread form over its entire length, e.g. with reference to the profiling and/or thread pitch thereof. At the coronal end, the dental implant can comprise a thread-free portion such that the threaded portion is attached to the thread-free portion in the apical direction.

In order to reduce the load on the material in the region of an anti-rotation element realized in the blind bore, the prior art often dispenses with the realization of an outer screw thread (which serves for primary or immediate anchoring of the implant in a jaw bone). As the ribs, which protrude according to the invention from the inner shell surface of the blind bore, however, do not require any reduction in the wall thickness in the region of the second anti-rotation element, this allows for a realization of a screw thread on the outside of the anti-rotation element without any stability losses for the dental implant. Irrespective of the presence of a thread-free portion on the coronal end of the dental implant, the second anti-rotation element is arranged, in a preferred manner, completely in the region of the threaded portion. For this reason, the threaded portion can also extend up to the coronal end of the dental implant.

In order to ensure as constant a wall thickness as possible in the region of the second anti-rotation element, the dental implant is realized in a preferred manner cylindrically, in particular circular cylindrically, in the region of the anti-rotation element. In particular when the dental implant comprises, for example, a cylindrical basic form which is tapered toward the apical end, the second anti-rotation element is arranged in a preferred manner in a cylindrical region with the widest possible diameter.

In a particularly preferred manner, the dental implant and the abutment are produced using an injection molding method. This makes it possible, in particular, to realize the anti-rotation elements and/or an internal thread provided in the blind bore already in the molding process. The ribs, grooves and/or any threaded elements do not have to be worked, e.g. milled, subsequently in this respect into the ceramic material, which reduces both the risk of damage to the ceramic components during post-processing and the production complexity. Specifically, the reduced wall thickness in the region of the grooves of the first anti-rotation element is far less of a problem in the case of production using an injection molding process than would be the case if the groove had to be worked subsequently into the ceramic material. In addition, an optimum fit of corresponding elements, such as grooves and ribs, can be ensured in the case of injection molding.

As mentioned above, according to the invention, the grooves are open toward the proximal end, which means that they extend either up to the proximal end of the abutment or open out proximally into a (hollow) cylindrical end portion, the outside diameter of which is smaller than that of the first basic body.

The connecting portion of the abutment extends in the distal direction in a preferred manner up to a circumferential shoulder which, in the connected state of the implant system rests on the coronal end of the dental implant and, as a result, surrounds the opening of the axial blind bore preferably in a sealing manner.

The connecting portion extends in the proximal direction in a preferred manner up to a ring-shaped end face which is delimited on the outside by a circumferential, in a preferred manner rounded end edge. The advantage of a rounded end edge is that, once the connecting portion is inserted into the axial blind bore, said rounded edge does not abut against the inner wall of the blind bore and also, where forces act at an angle on the abutment and the abutment tips minimally as a result with reference to the central longitudinal axis of the dental implant, it is not pressed against the inner wall of the blind bore. Load damage to the ceramic components can be avoided in this way.

According to a preferred embodiment, the head region of the abutment is realized in a substantially cylindrical manner or in the shape of a truncated cone, other, e.g. non-rotationally symmetrical forms, also being easily realizable. In a preferred manner, on its circumferential surface the abutment comprises a region with notches or an external thread in order to fasten a prosthetic element to the abutment. In addition, in a preferred manner, a further anti-rotation element for the prosthetic element is realized proximally of the notches or of the external thread, said further anti-rotation element being able to be in the form, for instance, of one cam or multiple cams.

In addition, in a preferred manner, the abutment comprises a transition portion which lies between the head part and the connecting portion and which is realized, in a particularly preferred manner, in the form of a truncated cone. A ring-shaped platform, which extends radially to the longitudinal axis of the abutment and is provided for supporting a prosthetic element, for instance a crown element, is realized in a preferred manner distally of the transition portion.

With reference to the material of the ceramic components of the implant system, in a preferred manner both the dental implant and the abutment are produced from zirconium oxide ceramic, in a particularly preferred manner from (yttrium) stabilized zirconium oxide ceramic. Zirconium oxide ceramic and in particular yttrium-stabilized zirconium oxide ceramic is particularly advantageous on account of its coloring and stability. In addition, they show excellent biocompatibility and a long service life in a moist warm environment, as is the case in the mouth area. However, other ceramics can also be used. As a result of choosing suitable stabilizing agents, such as, for example, yttrium oxide, cerium oxide, calcium oxide, magnesium oxide and/or erbium oxide, both the hardness and the color of the ceramic material can be matched to the individual requirements of the future wearer. Mixtures of ceramics can also be used for this purpose.

In a preferred manner, the two ceramic components of the implant system are realized integrally, i.e. in one piece from a composite material in order, ideally, to avoid boundary surfaces on which bacteria can collect and multiply. In addition, the number of parts which cooperate with one another and have to be matched to one another is reduced as a result.

The invention is described in detail by way of the accompanying figures, in which:

FIG. 1 shows a side view of an implant system according to a first embodiment of the invention;

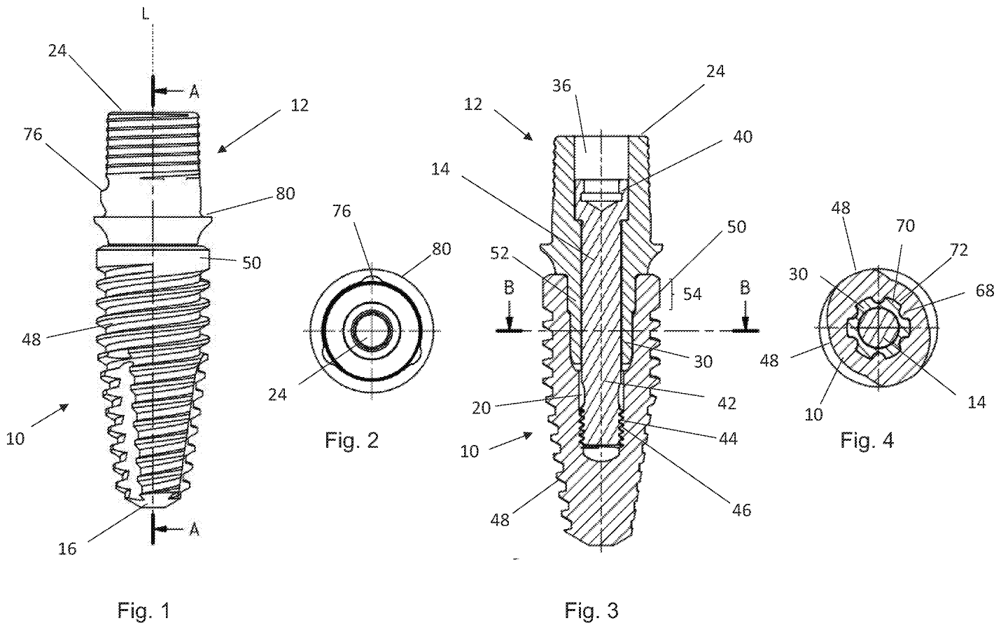

FIG. 2 shows a top view of the implant system according to FIG. 1;

FIG. 3 shows a section through the implant system according to FIG. 1 along a longitudinal center axis A-A;

FIG. 4 shows a section through the implant system according to FIG. 3 along a plane B-B perpendicular to the longitudinal center axis;

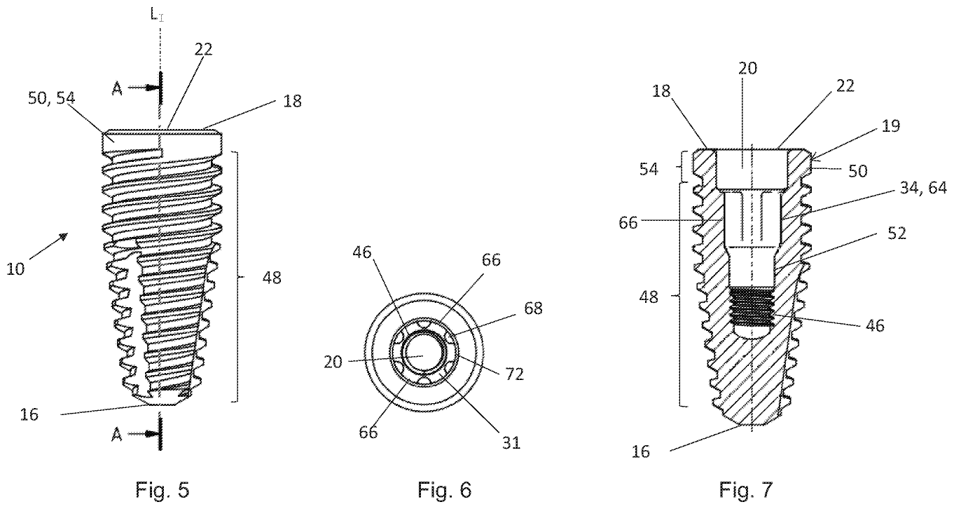

FIG. 5 shows a side view of the dental implant according to FIG. 1 on its own;

FIG. 6 shows a top view of the dental implant according to FIG. 5;

FIG. 7 shows a section through the dental implant according to FIG. 5 along the longitudinal center axis A-A;

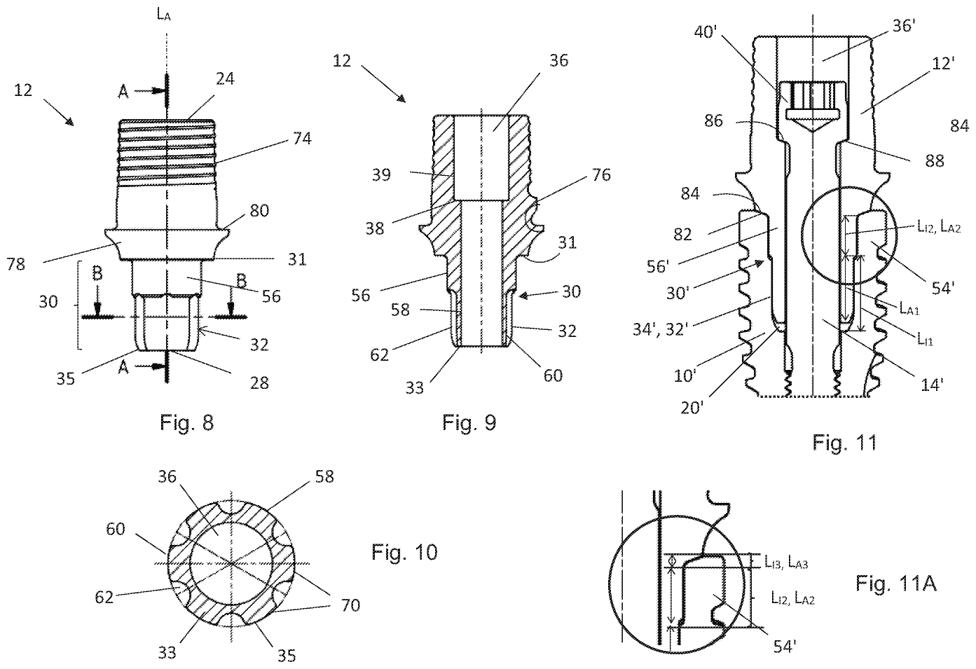

FIG. 8 shows a side view of the abutment according to FIG. 1 on its own;

FIG. 9 shows a section through the abutment according to FIG. 8 along the longitudinal center axis A-A;

FIG. 10 shows a section through the abutment according to FIG. 8 along the plane B-B;

FIG. 11 shows a section through an implant system according to an alternative embodiment; and

FIG. 11a shows an enlarged view of a detail of FIG. 11.

FIG. 1 shows an embodiment of an implant system according to the present invention. The implant system includes a dental implant 10 and an abutment 12 produced from a ceramic material, both of which are connected together in a sturdy manner by means of a connecting screw 14 (see FIG. 3). The ceramic components 10, 12 of the implant system, i.e. the dental implant 10 and the abutment 12, are produced in a preferred manner using an injection molding process. An yttrium-stabilized and/or cerium-stabilized zirconium oxide ceramic is used preferably in its production. As an alternative to this, other biocompatible ceramic materials that are suitable for use in the dental field are also conceivable.

The dental implant 10 is provided for anchoring in a jaw bone and extends along a longitudinal axis L_{I} from an apical end 16 to a coronal end 18. In addition, it comprises a blind bore 20 which is open toward the coronal end 18, extends coaxially to the longitudinal axis L_{I} of the dental implant 10 and has a coronal opening 22 (see FIG. 3) into which the abutment 12 is inserted. The blind bore 20 is realized in a cylindrically stepped manner and includes a ring-shaped shoulder surface 23 (see FIG. 7) which serves for support of the abutment 12.

The abutment 12, which is shown as a whole unit in FIGS. 3, 8 and 9, comprises a distal end 24 with a head portion 26 for receiving a prosthetic element, e.g. a tooth crown (not shown), and an oppositely situated proximal end 28 with a connecting portion 30. The connecting portion 30 is provided for insertion into the blind bore 20 of the dental implant 10 and comprises a ring-shaped shoulder 31 (see FIG. 8) which, in the connected state of the implant system, is supported on the shoulder surface 23 of the dental implant 10 (see FIG. 3). In the proximal direction, the connecting portion 30 extends up to a ring-shaped end face 33 (see FIG. 10) which is delimited on the outside by a circumferential end edge 35. Said end edge 35 is rounded such that, in the connected state of the implant system, it does not come into contact with the inner wall of the axial blind bore 20 (see FIG. 3). On the outside, the connecting portion 30 additionally comprises a first anti-rotation element 32, which is described in detail in conjunction with FIGS. 8-10. The first anti-rotation element 32 is intended to interact with a complementary second anti-rotation element 34, which is realized in the blind bore 20 of the dental implant 10, in order to prevent the abutment 12 rotating about a longitudinal axis once it has been inserted into the blind bore 20 of the dental implant 10.

The abutment 12 additionally includes a through-bore 36, which extends from the distal head portion 26 up toward the proximal end 28 (see also FIG. 9) and which consequently penetrates the abutment 12 completely and serves for receiving a connecting screw 14 (see FIG. 3). In the embodiment shown, the through-bore 36 extends along the longitudinal axis L_{A} of the abutment 12 and is aligned with the longitudinal axis L_{I} of the dental implant 10. In the event of an angled abutment (not shown), the through-bore 36 is certainly also in alignment, as a rule, with the longitudinal axis L_{I} of the dental implant 10, but is positioned with reference to the longitudinal axis L_{A} of the abutment 12 in such a manner that it encloses an angle with this latter. In a middle region of the through-bore 36, the abutment 12 comprises a shoulder 38 which serves as contact surface for the underside of a screw head 40 of the connecting screw 14 (see FIG. 3). The diameter of the through-bore 36 is narrower proximally of the shoulder 38 than in a region 39 which is located distally of the shoulder 38.

The connecting screw 14 is produced, as a rule, from metal, in a preferred manner titanium, which is advantageous as regards stability. As can be seen the best from FIG. 3, the connecting screw 14 includes a distal screw head 40 and a shank 42 with an external threaded portion 44 located proximally. The diameter of the shank 42 is smaller than that of the through-bore 36. The diameter of the screw head 40 is smaller than the diameter of the region 39 which adjoins the shoulder 38 of the abutment 12 distally and is greater than that of a region of the through-bore 36 which adjoins the shoulder 38 proximally. Consequently, the connecting screw 14 can only be inserted into the through-bore 36 until the underside of the screw head 40 rests on the shoulder 38. The length of the connecting screw 14 is chosen such that the proximal external threaded portion 44, once the connecting screw 14 has been inserted into the abutment 12 (until the screw head 40 rests on the shoulder 38) projects proximally out of the through-bore 36. Thus, the external threaded portion 44 can be screwed in a known manner into an internal threaded portion 46, which is arranged apically of the second anti-rotation element 34 in the blind bore 20 of the dental implant 10, in order to connect the abutment 12 to the dental implant 10 in a reversible manner.

As can be seen best in FIGS. 1 and 5, on the outside the dental implant 10 comprises a threaded portion 48, which is self-tapping in a preferred manner and extends in large parts over the length of the dental implant 10. On the coronal end 18, the dental implant 10 includes a thread-free portion 50. As the second anti-rotation element 34 also serves, as a rule, as a contact point for an insertion tool for screwing the dental implant 10 into the jaw bone, it is not realized directly in the coronal end region 19, but further apically in the blind bore 20 (see FIG. 7). Thus, the thin-walled coronal end region 19 is largely protected from the torsional forces which arise when the dental implant 10 is screwed-in. In the case of the embodiment shown in FIG. 7, the second anti-rotation element 34 is arranged further down in the blind bore for this reason and consequently completely in the region of the threaded portion 48.

With regard to the greatest possible reduction in the forces acting on the material in the region 19 of the second anti-rotation element 34, a thread-free, hollow cylindrical portion 52 is arranged between the second anti-rotation element 34 and the internal threaded portion 46 that is realized in the blind bore 20, (see FIG. 7). As a result, stresses, which can arise when the connected screw 14 is screwed-in, are kept away as much as possible from the region 19 of the second anti-rotation element 34. In addition, the internal threaded portion 46 is positioned exclusively in the lower, i.e. apical, half of the blind bore 20 in order to relieve the coronal end region 19 of the implant 10.

On the coronal end 18, the dental implant 10 additionally comprises a hollow cylindrical end portion 54 which extends substantially up to the coronal end 18 of the axial blind bore 20 and to which the second anti-rotation element 34 is attached in the apical direction. In a complementary manner to the end portion 54, a hollow cylindrical neck portion 56 is realized distally of the first anti-rotation element 32 of the abutment 12, the neck portion 56 being positioned inside the end portion 54 in the connected state of the implant system components 10, 12 (see FIG. 3). As a consequence, the inner radius of the end portion 54 is realized in a complementary manner to the outer radius of the neck portion such that a precisely-fitting connection between abutment and implant is achieved. In the case of said embodiment, the second anti-rotation element 34 consequently does not extend up to the coronal end 18 of the dental implant 10, but simply up to the hollow cylindrical end portion 54. As a result, it is ensured that the coronal anti-rotation element 34 is positioned sufficiently deeply in the blind bore 20, i.e. sufficiently far away from the coronal end 18 in order to keep the forces acting on the material in the coronal end region 19 as small as possible.

The form of the anti-rotation elements 32, 34 is advantageous with regard to increased stability and reduced fracture susceptibility of the implant system components that are to be connected (that is to say of the dental implant 10 and of the abutment 12):

As can be seen the best in FIGS. 8, 9 and 10, the first anti-rotation element 32 of the abutment 12 includes a hollow cylindrical first basic body 58 with an outer shell surface 60 and additionally comprises multiple grooves 62 which extend in the longitudinal direction L and, proceeding from the outer shell surface 60, project into the first basic body 58. Said grooves 62 are open toward the proximal end 28 of the abutment 12 and, in the embodiment shown, extend up to the proximal end 28 of the abutment 12.

The second anti-rotation element 34 of the dental implant also comprises, correspondingly, a hollow cylindrical second basic body 64 with an inner shell surface 66 and with multiple ribs 68 which extend in the longitudinal direction and project, proceeding from the inner shell surface 66, into the interior of the axial blind bore 20 (see FIG. 6). In the connected state, the ribs 68 of the dental implant 10 engage in the grooves 62 of the abutment and form an anti-rotation connection between the two ceramic components 10, 12 of the implant system (see FIG. 3).

The advantage of the design according to the invention of the anti-rotation device produced from interlocking grooves 62 and ribs 68 is that, in contrast to otherwise often used anti-rotation devices with a polygonal cross section, it is possible to dispense with the realization of sharp edges and corners and, as a result, to avoid load peaks. In addition, two adjacent grooves 62 or ribs 68 are spaced apart from one another in each case by portions 70/72 of the inner shell surface 60/68 of the corresponding hollow cylindrical basic body 58/64 and the width of the portions 70/72 measured in the circumferential direction is greater than the width of the grooves 62 or ribs 68. As a result, the stability bestowed by the hollow cylindrical basic body 58/64 in the region of the corresponding anti-rotation element 32/34 is maintained.

The downwardly open design of the grooves 62 extending up to the proximal end 28 enables simple insertion of the ribs 68 of the second anti-rotation element 34 into the grooves 62 when the connecting portion 30 of the abutment is inserted into the blind bore 20 of the dental implant 10.

As can be seen well in FIGS. 4 and 6, the grooves 62 and ribs 68 each comprise an approximately semi-circular cross section. Both the grooves 62 and the ribs 68 are consequently in the form of a cylinder which is cut in the longitudinal direction, the concavely or convexly curved basic area of which is formed by a portion of the respective shell surface 60/68 of the associated basic body 58/64. Said form allows the forces acting on the anti-rotation elements 32/34 to be distributed in a homogeneous manner.

In a possible embodiment, the second anti-rotation element 34 extends over a length of approximately 2 mm and comprises six ribs 68 which are uniformly spaced apart from one another in the circumferential direction (see FIG. 6). The first anti-rotation element 32 comprises, correspondingly, six grooves 62 which are uniformly spaced apart from one another in the circumferential direction (see FIG. 10). It is, however, also conceivable to provide a smaller number of ribs 68 than grooves 62. The ribs 68 define the number of possible alignment possibilities for the abutment 12 in relation to the dental implant 10. With reference to the stability of the anti-rotation device, it has been shown that a higher number of grooves 62 or ribs (three or more) in place of just one or two grooves 62 or ribs 68 is advantageous. In addition, the grooves 62 in the embodiment shown are realized in a relatively narrow manner in order to impair the wall thickness and consequently the stability of the wall in the region of the first anti-rotation element as little as possible. It has also been shown that a width-length ratio of the grooves 62 or ribs 68 of at least 1:3 is advantageous with reference to the stability of the anti-rotation device, on the one hand, and to the fracture strength of the material in the region of the anti-rotation elements 32, 34 on the other hand.

As can be seen from FIGS. 8 and 9, the head portion 26 of the abutment 12 is realized in a substantially cylindrical manner and on its circumferential surface comprises a region with an external thread 74. The external thread 74 serves for fastening a prosthetic element, such as a crown or a bridge element (not shown). Said external thread 74 can also be replaced by notches or ribs as an alternative to this. A further anti-rotation element in the form of three cams 76, which are uniformly spaced apart from one another in the circumferential direction, is realized proximally of the external thread 74 (see FIG. 2). Thanks to the cams 76, a prosthetic element is able to be fastened non-rotatably on the abutment 12.

A truncated cone-shaped transition portion 78, to which a ring-shaped platform 80 is attached distally, is realized between the head portion 26 and the connecting portion 30. The ring-shaped platform 80 extends radially with respect to the longitudinal axis L_{A} of the abutment 12 and serves as a contact surface for a prosthetic element.

FIG. 11 shows further an alternative embodiment to the implant system shown in FIG. 3. Most features are the same in both embodiments. The implant system according to FIG. 11 differs from the embodiments shown in FIGS. 1 to 10 particularly in that the blind bore 20′ of the dental implant 10′ further comprises a tapered section 82, which is provided coronally of the hollow cylindrical end portion 54′ and which has a diameter that increases in the coronal direction. In addition, the connecting portion 30′ of the abutment 12′ comprises a complementary tapered section 84 that is located coronally of the cylindrical neck portion 56′, such that the tapered surfaces 82, 84 contact each other after full insertion of the connecting portion 30′ of the abutment 12′ into the blind bore 20′ of the implant 10′. Thanks to the tapered contact surfaces 82, 84 the transmission of forces from the abutment 12′ to the dental implant 10′ is improved. The tapered contact surfaces 82, 84 also assist centering of the abutment 12′ when the latter is inserted into the blind bore 20′ of the dental implant 10′.

The taper angle of the tapered sections 82, 84 is usually 5° to 35°, in the shown example it is about 20°.

The axial length L_{I3} of the tapered section 82 of the blind bore 20′ of the implant 10′ is smaller than the axial length L_{I2} of the hollow cylindrical end portion 54′, and also than the axial length L_{I1} of the coronal anti-rotation element 34′. L_{I2} is also smaller than L_{I1}. In the shown embodiment, the length L_{I3} is about ⅕ of the length L_{I2}.

Similarly, the axial length L_{A3} of the tapered connection portion 84 of the abutment 12′ is smaller than the axial length L_{A2} of the cylindrical neck portion 56′, and also than the axial length L_{A1} of the first anti-rotation element 32′. L_{A2} is also smaller than L_{A1}. In the shown embodiment, the length L_{A3} is about ⅕ of the length L_{A2}.

Comparing the embodiments of the dental implant 10 and the abutment 12 shown in FIGS. 1 to 10 with the embodiment of the dental implant 10′ it is evident that the axial length L_{I2} of the hollow cylindrical end portion 54′ is reduced, since the tapered section 82 replaces part of the hollow cylindrical end portion 54′. For that reason, the axial length L_{I2} of the hollow cylindrical end portion 54′ or the axial length L_{I2} of the neck portion 56′ can in such embodiments be less than half of the length L_{A1}, L_{I1} of the first and the second anti-rotation element 32′, 34′, respectively. However, the combined axial lengths L_{I3} and L_{I2} of the tapered section 82 and the hollow cylindrical end portion 56′ of the blind bore 20′ are at least half as long as the axial length L_{I1} of the second anti-rotation element 34′. Similarly, the combined axial lengths L_{A3} and L_{A2} of the tapered section 82 and the neck portion 56′ of the abutment 12′ are at least half as long as the axial length L_{A1} of the first anti-rotation element 32′.

As can also be seen in FIG. 11, the connecting screw 14′ has a screw head 40′ with a conically tapered underside 86. Said underside 86 abuts or rests on a complementary tapered screw seat 88 in the bore 36′ of the abutment 12′. The connection of the tapered underside 86 with the tapered screw seat 88 facilitates the force transmission and further helps the transmission of the forces from the screw 14′ to the abutment towards the tapered sections 82, of the blind bore 20′ and of the connecting portion 30′, respectively.

The taper angle on the screw head 40′ is usually about 10° to 70°. In the shown embodiment the head has a taper angle of about 20°. Alternatively, a taper angle of about 60° is particularly preferred.

1. An implant system including a dental
implant and an abutment produced from a ceramic material,
wherein the dental implant extends along a longitudinal center axis L_{I} from an apical end toward a coronal end and comprises an axial blind bore which is open toward the coronal end and, on an outer surface, a screw thread for anchoring in a jaw bone,
the abutment comprises a distal end with a head portion for receiving a prosthetic element, a proximal end, which is situated opposite the distal end and has a connecting portion for insertion into the blind bore of the dental implant, and a through-bore which extends from the distal end to the proximal end for receiving a connecting screw,
a first anti-rotation element is realized on the outside of the connecting portion and a second anti-rotation element, which is complementary to said first anti-rotation element, is realized on the inside in the blind bore,
wherein:
the first anti-rotation element of the abutment includes a hollow cylindrical first basic body with an outer shell surface and with multiple grooves which extend in the longitudinal direction and, proceeding from the outer shell surface, project into the first basic body, wherein the grooves are open toward the proximal end, and
the second anti-rotation element of the dental implant includes a hollow cylindrical second basic body with an inner shell surface and multiple ribs which extend in the longitudinal direction and, proceeding from the inner shell surface, project into the axial blind bore.

2. The implant system as claimed in claim 1, wherein the dental implant includes an internal threaded portion, which is arranged apically of the second anti-rotation element, for connection to a connecting screw.

3. The implant system as claimed in claim 1, wherein two adjacent grooves or ribs are spaced apart from one another in each case portions of the outer or inner shell surface of the corresponding hollow cylindrical basic body and the width of the portions measured in the circumferential direction is greater than the width of the grooves or ribs.

4. The implant system as claimed in claim 1, wherein the grooves extend substantially up to the proximal end of the abutment.

5. The implant system as claimed in claim 1, wherein the grooves and the ribs each comprise a cross section that is in the shape of a segmental arch at least in portions.

6. The implant system as claimed in claim 1, wherein the cross-sectional surface of the grooves and ribs comprise at least one base line which is formed through the outer or rather inner shell surface of the corresponding hollow cylindrical basic body of the respective anti-rotation element and the end points of which are connected by means of a connecting line which is accurate at least in portions.

7. The implant system as claimed in claim 6, wherein the connecting line includes a circular arc with a uniform radius.

8. The implant system as claimed in claim 1, wherein the grooves or the ribs comprise a width-length ratio of between 1:3 and 1:6.

9. The implant system as claimed in claim 1, wherein the axial blind bore comprises a hollow cylindrical end portion coronally of the second anti-rotation element and, distally of the first anti-rotation element, the abutment comprises a complementary hollow cylindrical neck portion which is arranged inside the end portion once the connecting portion has been inserted into the blind bore.

10. The implant system as claimed in claim 9, further comprising a tapered section that is provided coronally of the hollow cylindrical end portion within the blind bore of the dental implant and has a diameter that increases in the coronal direction, and the implant system further comprising a complementary tapered section that is provided coronally of the cylindrical neck portion on the connecting portion of the abutment, wherein the surfaces of the two tapered sections contact each other after full insertion of the connecting portion of the abutment into the blind bore of the implant.

11. The implant system as claimed in claim 10, wherein the hollow cylindrical end portion of the dental implant extends substantially up to the coronal end of the axial blind bore and its length, is at least half as long as the length of the second anti-rotation element.

12. The implant system as claimed in claim 1, wherein the connecting portion extends in the proximal direction up to a ring-shaped end face which is delimited on the outside by a circumferential rounded end edge.

13. The implant system as claimed in claim 1, wherein the connecting portion extends in the distal direction up to a circumferential shoulder which, in the connected state of the implant system, rests on the coronal end of the dental implant and, as a result, surrounds the opening of the axial blind bore.

14. The implant system as claimed in claim 1, wherein the second anti-rotation element is arranged completely in the region of the threaded portion.

15. The implant system as claimed in claim 1, wherein the dental implant and/or the abutment are produced using a (powder) injection molding method.

16. The implant system as claimed in claim 1, wherein the first anti-rotation element and the second anti-rotation element include an identical number of grooves or ribs.
